(19) United States
(12) Patent Application Publication
(10) Pub. No.: US 2009/0069757 A1
(43) Pub. Date: Mar. 12, 2009

(54) ELASTOMERIC SEAL FOR USE WITHIN A CATHETER COMPONENT

(75) Inventors: John I. Muri, Laguna Niquel, CA (US); Bryan Knodel, Flagstaff, AR (US); Gaylene Fisch, San Diego, CA (US)

Correspondence Address: JAMES D. LEIMBACH, 3103 EVENING WAY, UNIT E, LA JOLLA, CA, 92037, US

(73) Assignee: MEDCO INTERNATIONAL, LLC, Carson City, NV (US)

(21) Appl. No.: 12/205,888

(22) Filed: Sep. 7, 2008

Related U.S. Application Data
(60) Provisional application No. 60/967,788, filed on Sep. 8, 2007.

(51) Int. Cl. A61M 25/00; B23P 11/00

(52) U.S. Cl. 604/256; 29/428

(57) ABSTRACT

A sealing engagement between components in a catheter system is made using an elastomeric material. The sealing engagement is formed in a manner that allows a catheter needle to pass through a passageway in the center of the elastomeric material maintaining the sealing engagement between the components of the catheter system. The elastomeric material is shaped to fit circumferentially into one of the components and shaped to mechanically assist the passageway to close up after a needle passes through the passageway during retraction. The elastomeric material remains in a stationary position as the needle is retracted. One or more structures can be integrated within the elastomeric material to provide for compression and to prevent deformation of the passageway.

### BACKGROUND OF THE INVENTION

1. Field of the Invention

The present invention relates to catheter systems, and more particularly, to methods and devices for creating a sealing engagement between components within a catheter system.

2. Description of the Prior Art

Catheters allow for fluid communication with patients to introduce fluids into a patient and remove blood from a patient. It is not uncommon for catheters to be the over the needle type wherein a needle and catheter are provided in coaxial arrangement. The needle is used as an introducer to provide a point of entry for the catheter into a patient by inserting the needle into the patient's vein and then sliding the catheter over the needle into the vein of the patient while removing the needle from the vein of the patient. Providing isolation between the fluids that flow through lumens within the catheter system and ambient conditions is important. Specifically, major concerns exist regarding the spread of blood borne pathogens of various types and for isolating the fluid exchange with a patient from clinicians that are in the vicinity. Still other concerns relate to contamination of catheter and the needle from ambient conditions.

Current existing catheter designs provide isolation between chambers of a catheter system and ambient surroundings. Among these are designs are various seals and O-rings. One prior art teaching, US Published Patent Application No. 2007/0083157, teaches a seal that is retained within a chamber. This seal provides a passageway through which the catheter needle can be removed. Upon removal of the catheter needle, an inherent force is applied causing the passageway to close. This seal allows for reopening through an elaborate design that is complicated. While effective in sealing this interior lumen from ambient surroundings under predetermined conditions, the expense in producing this seal is a shortcoming. The seal taught by US Published Patent Application No. 2007/0083157 is not intended to create a sealing engagement that once the needle is removed will not be reopened. There is a need for a seal that allows a needle to be withdrawn, retains the sealing engagement after withdrawal and is cost effective to produce. Therefore, there remains a need within the art of catheters for a sealing engagement that allows a catheter needle to pass, be withdrawn and provides isolation for chambers within a catheter system in both cases.

There are numerous sealing engagements taught by the prior art. A typical problem that exits with these prior art sealing engagements is that they are overly complicated resulting in more expensive resulting in end products that are more expensive and less reliable. Therefore, there remains a need within the art of catheters for a sealing engagement that use fewer parts in a simpler design that is more reliable.

### SUMMARY OF THE INVENTION

Embodiments described herein address the shortcomings of the prior art by providing a seal made from an elastomeric material that is used to create a sealing engagement between components in a catheter system. The seal can be designed to allow a catheter needle to pass through the center of the seal while maintaining the sealing engagement between the components of the catheter system. The seal used to create the sealing engagement can be shaped to circumferentially fit into a component and designed with an aperture through which the needle can pass that closes after the needle is withdrawn. The shape of the seal can assist closing the aperture after a needle is removed from the middle. The seal can be formed with a compliant “nipple” to facilitate complete closure. The nipple permits the use of softer materials while maintaining sealing performance. The uses of softer materials result in less drag allowing the device to function with a lower force spring. Lower force springs are more compact leading to a shorter overall length for the catheter device. The seal remains stationary in position within the component as the needle is removed.

An object of an embodiment is to provide a seal within an L-tube component of a catheter system. The L-tube has a distal end closest to the patient and a proximal end closest to the clinician that is performing the canulation of the patient. The distal end of the L-tube can have a catheter hub attached thereto with a needle used as an introducer into venous tissue of the patient. The proximal end of the L-tube will have a safety tube attached that the needle can be retracted into. The seal can be fit into the proximal end of the L-tube and the needle will pass through the center of the seal. The seal is compressed into the distal end of the L-tube such that a sealing engagement is created between the L-tube and the safety tube. The seal can contain a support structure to prevent the passageway within the seal through which the needle traverses from being damaged by the weight of the needle or movement of the needle during retraction. The seal can be designed to fit into the proximal end of the L-tube. The seal is shaped such that the passageway through which the needle passes will be closed after the needle is withdrawn. The shape of the seal inherently assists closing the aperture after a needle is removed. The seal has a compliant “nipple” that is used to facilitate complete closure. The nipple permits the use of softer materials while maintaining sealing performance. The uses of softer materials result in less drag allowing the device to function with a lower force spring. Lower force springs are more compact leading to a shorter overall length for the catheter device. The seal remains stationary within its position in the component as the needle is removed.

It is an object of another embodiment to provide a catheter system wherein a sealing engagement exists between catheter components both before and after canulation of the patient. Forming a seal and providing a center conical section through which a catheter needle traverses to provide the sealing engagement between components. An annular ridge is formed on the end of the seal where the conical section is formed compresses the center conical section while the seal is within contained within the component. The seal is compressed within the component such that the annular ridge presses inward on the conical section. The inward pressure is such that the needle can slide through a passageway in the conical section and the conical section will close upon removal of the needle.

It is further an object to provide an embodiment that provides a seal capable of providing fluid isolation between catheter components while a catheter needle is being used as an introducer into the venous tissue of a patient and after the catheter needle has been withdrawn that also has support structure associated with the seal to prevent the catheter needle from deforming the seal from the weight of the needle upon the seal or deformation occurring during withdrawal of the catheter needle.

It is an object of another embodiment to provide a seal with at least one annular ridge that allows a sealing attachment between two components within a catheter system. The seal is compress fitted into one of the components. The compression applies a circumferential force to a catheter needle that traverses the center of the seal. At least one end of the seal contains a formation that seals the passageway traversed by the needle once the needle is removed. The seal has a compliant “nipple” that is used to facilitate complete closure. The nipple permits the use of softer materials while maintaining sealing performance. The uses of softer materials result in less drag allowing the device to function with a lower force spring. Lower force springs are more compact leading to a shorter overall length for the catheter device.

It is an object of another embodiment to provide a seal that can be placed in a proximal portion of a first component of an intravenous catheter such that fluid is prevented from passing through to the distal side of a second component that is attached to the first component, thus preserving a sealing engagement between the first and second component. The seal can house a needle that is part of an intravenous catheter device. The needle passes through the middle of this elastomeric material. The needle will be mechanically supported on both the proximal and distal ends outside of the seal so that there is no pressure placed on the seal itself while the needle rests in it. This seal is shaped to fit circumferentially into a component and has a shape that mechanically assists the hole to close up after a needle is removed from the middle. The seal has a compliant conical formation that is used to facilitate complete closure. The conical formation permits the use of softer materials while maintaining sealing performance. The uses of softer materials result in less drag allowing the device to function with a lower force spring. Lower force springs are more compact leading to a shorter overall length for the catheter device. The seal is intended to remain in a stationary position once the needle is removed.

It is an object of another embodiment to provide a seal with at least one annular ridge that can be used to form a sealing attachment between two components within a catheter system. The seal is compress fitted into one of the components. The compression applies a circumferential force to a catheter needle that traverses the center of the seal. At least one end of the seal contains a portion that closes upon the passageway traversed by the needle once the needle is removed. The portion is a conical protrusion that is used to facilitate complete closure. The use of a conical protrusion permits the use of softer materials while maintaining sealing performance. The uses of softer materials result in less drag allowing the device to function with a lower force spring. Lower force springs are more compact leading to a shorter overall length for the catheter device.

### BRIEF DESCRIPTION OF THE DRAWINGS

FIG. 1a is a perspective view of an elastomeric material having a circumferential ridge and a conical protrusion formed in a recess at the center of one end used to form a seal with a catheter component;

FIG. 1b is a perspective view of an elastomeric material for a catheter component having one end that is solid through which a passageway is formed for a catheter needle to traverse;

FIG. 2 is a diametrical view illustrating various views of the seal of FIG. 1 including a cross sectional view;

FIG. 3a is a perspective view of a catheter component from the proximal end that houses the elastomeric material of FIG. 1;

FIG. 3b is a perspective view of a catheter component from the distal end that will contain a catheter hub; and

FIG. 4 is a cross sectional view of an embodiment for a sealing engagement that employs two seals made of an elastomeric material.

### DETAILED DESCRIPTION

As used in this document, the term seal refers to any single device or combination of devices that can create a sealing relationship between two portions of one component or between two different components. The terms proximal or proximally are intended to reflect being closer to the clinician and farther away from the patient. The terms distal or distally are intended to reflect being farther away from the clinician and closer to the patient.

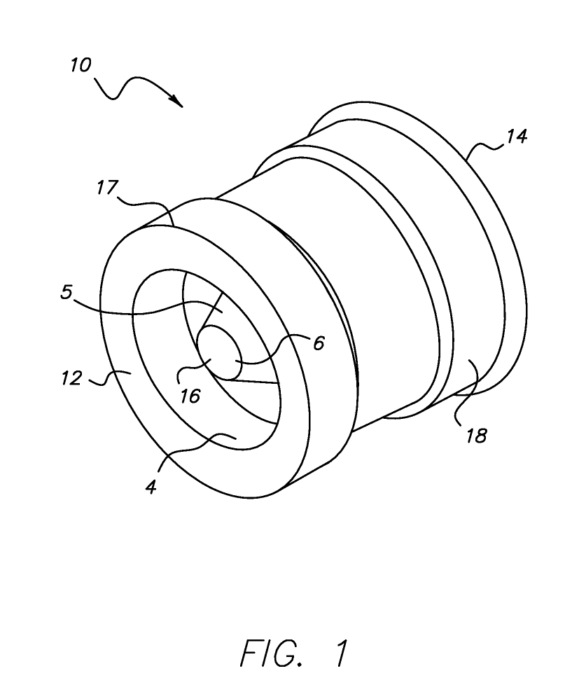

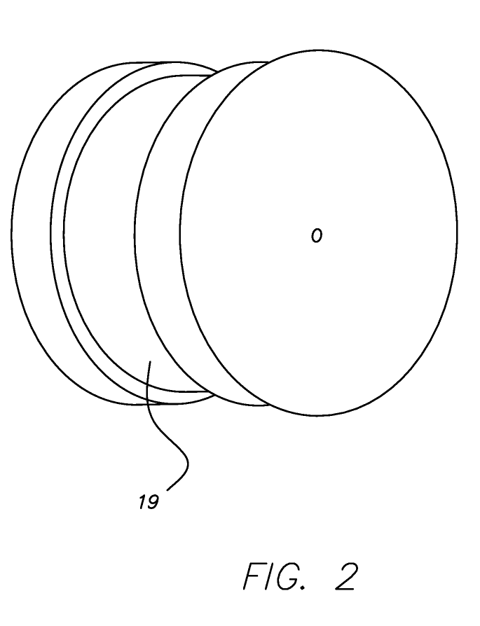

Referring to FIG. 1a, an embodiment is illustrated for a seal 10 that is constructed from an elastomeric material that is used to create a sealing engagement between components in a catheter system. The elastomeric material can be either silicon or polyisoprene or any combination, thereof, having hardness in a range of 15 to 65 ShoreA. Other embodiments can employ an elastomeric material made from either silicon, latex or polyisoprene or any combination, thereof, with hardness in the range of 35 to 45 ShoreA. The seal 10 is formed with a proximal end 12 that is closest to the clinician and a distal end 14 that is closest to the patient. Seal 10 is designed to allow a catheter needle (not shown) to pass through a central passageway 16 that traverses from the proximal end 12 to the distal end 14 of the seal 10. The seal can be placed within a first catheter component (not shown) with a catheter needle traversing passageway 16. The seal 10 maintains a sealing engagement between the first component into which it is placed and a second component of the catheter system. The seal 10 contains at least one of annular ridges 17, 18 that can be formed near the proximal end 12 and/or distal end 14, respectively. Annular ridges 17, 18 apply an axial force on a catheter component allowing the seal 10 to create a sealing engagement once the seal 10 is placed into a compatible portion of the catheter component. The seal 10 can include a recess 4 at the proximal end 12 and a conical protrusion 5 formed within the recess 4 such that the axial force will close the passageway 16 once the catheter needle is withdrawn. The shape of the conical protrusion 5 of seal 10 assists closing the passageway 16 after the catheter a needle is removed. The seal 10 remains stationary within its position in the first component as the catheter needle is removed.

The internal passageway 16 can be constructed in various ways. One method of construction is to use a trocar tipped pin (3 cutting edges) to cut the passageway 16 within the elastomeric material used for the seal 10. Another method is to employ point end piercing which uses a single pin like cutting edge. Yet another method of forming the internal passageway is to use a catheter needle to form the passageway 16 resulting in a sealed mounting for the catheter needle.

Another embodiment includes seal 10 with a nipple 6 formed on the conical protrusion 5 that is used to facilitate complete closure. The nipple 6 can be a separate material from that used to create the seal 10 which permits the use of softer materials for the nipple 6 while maintaining sealing performance of seal 10. The uses of softer materials result in less drag allowing the catheter needle to be withdrawn with a smaller force. A smaller force allows for a biasing device that is are more compact leading to a shorter overall length for the catheter device.

FIG. 1b illustrates a perspective view of the seal 10 viewed from the distal end 14. Seal 10 is designed to allow for insertion into the first component with annular ridges 17, 18 formed near proximal end 12 and distal end 14, respectively, to apply an axial compression force on the seal 10 once inserted into the first component creating a sealing engagement with the first component. The seal 10 as shown in FIG. 1b can be formed with a central annular region 19 between annular ridges 17, 18 to allow for easy insertion into the first component. A recess 4 formed at the proximal end 12 with a conical protrusion 5 formed within the recess 4 such that the axial force will close the passageway 16 once the catheter needle is withdrawn. The shape of the conical protrusion 5 of seal 10 assists closing the passageway 16 after the catheter a needle is removed. The seal 10 remains stationary within its position in the first component as the catheter needle is removed. Central passageway 16 that traverses from the proximal end 12 to the distal end 14 of the seal 10 can be seen in FIG. 1b from the distal end 14.

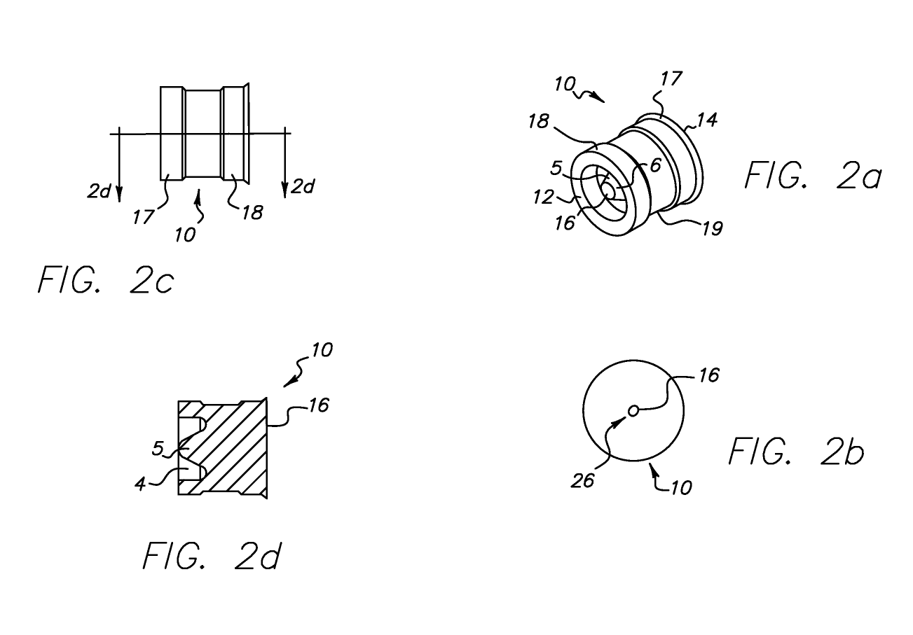

FIGS. 2a through 2d illustrate an embodiment for a seal used within a catheter system to create a sealing engagement between catheter components both before and after canulation of the patient. It should be understood that dimensions shown in FIGS. 2a through 2d are for a single embodiment and that these dimensions will change for different embodiments of catheter systems that may be used. FIG. 2a illustrates a seal similar to that shown in FIG. 1a and portions of the seal shown in FIGS. 2a-2d that have similar functions are identified by the same reference numeral. Seal 10 has proximal end 12 and distal end 14 with annular ridges 17 and 18, respectively. A central conical section 5 having passageway 16 through which a catheter needle can traverse provides for the sealing engagement between the component that seal 10 is inserted to and another component. The annular ridge 17 formed on the end of the seal where the conical section 5 is formed compresses the center conical section 5 while the seal 10 is within contained within the component (not shown). The seal 10 is compressed within the component such that the annular ridge 17 presses inward on the conical section 5. The inward pressure is such that the needle can slide through a passageway 16 in the conical section 5 and the conical section 5 will close upon removal of the needle. To facilitate the movement of the needle while under compression, nipple 6 is formed at the tip of conical section 5. Nipple 6 can be made from a very soft material to facilitate closure of passageway 16 once the catheter needle is removed from the passageway 16. Less drag on the movement of the catheter needle results during movement within the passageway 16 by using a softer material to make nipple 6, and possibly the entire conical section 5. Less drag allows for retraction of the catheter needle using a lower biasing force and a lower biasing force allows for the use of retraction device that are shorter, thereby, providing for a catheter device having a shorter overall length.

Referring to FIGS. 2c and 2d, an embodiment for seal 10 with at least one annular ridge allows for a continued a sealing attachment between two components within a catheter system. FIG. 2c shows a side view of seal 10 with ridges 17 and 18. A sectional view along line A-A′ is shown in FIG. 2d that illustrates conical section 5 within recess 4. The seal 10 is compress fitted into one of the components. The compression to annular ridge 17 applies a circumferential force to conical section 5 and to the passageway 16 at the proximal end 12 through which a catheter needle traverses. Having a formation such as conical section 5 on at least one end of seal 10 can assert a force on passageway 16 to create a sealing engagement around the needle and once the needle is removed continue the sealing engagement. Conical section 5 can be made from the same elastomeric material used to form seal 10.

In another embodiment, conical section 5 can be made of a softer material or have a compliant nipple 6 formed at the end of conical section 5 that facilitates complete closure of passageway 16 upon removal of the catheter needle. The use of softer materials for conical section 5 or nipple 6 maintains a sealing engagement while resulting in less drag caused by the sliding of the catheter needle, which in turn allows for the needle to be retracted using a lower biasing force. The biasing force can come from a spring or other biasing device. A lower biasing force yields more compact and shorter overall length for the catheter device. The remainder of seal 10 can be a stiffer material, although not necessarily so, to hold the seal in place in the component during retraction of the catheter needle.

FIG. 2d illustrates conical section 5 that can be used to provide additional support at the proximal end 12 of seal 10. Using a softer material that is highly resilient for conical section 5 can prevent deformation at the proximal end 12 of seal 10 by the resiliency of the material used. It further allows for a stiffer elastomeric material to be used elsewhere in seal 10 to prevent deformation.

Referring to FIG. 2a, nipple 6 can be constructed from a material intended to reduce deformation. Conical section 5 can be produced from a softer material to insure a strong seal along passageway 16 and nipple 6 can be a material that is stiff to prevent deformation from the weight of the catheter needle or to resist deformation once the needle is being retracted. By providing a structure on at least one end of the seal 10 that prevents deformation of passageway 16, the sealing characteristics of the seal 10 can be maintained.

Another embodiment provides a sealing engagement capable of fluidly isolation catheter components during the time that the catheter needle is being used as an introducer into the venous tissue of a patient until after the catheter needle has been withdrawn. A support structure associated with seal 10 can be provided on the proximal end 12 or the distal end of seal 10, or both. To prevent deformation in the passageway 16 portion of seal 10, the support structure needs to prevent the weight of the needle or sliding movements of the needle from pressing against the passageway. The use of a firm conical section 5 or nipple 6 can enhance the sealing characteristics of seal 10 by not allowing the catheter needle to deform the passageway 16 within seal 10 from the weight of the needle upon the seal or deformation occurring during withdrawal of the catheter needle. Referring to FIG. 2b, which shows the distal end of seal 10, there can also be a support structure 26 on the distal end of seal 10 to prevent deformation in the passageway 16. An annular ring made from a firm material can be placed into a recessed area, or a different material can be used. Additionally, a structure similar to that used on the proximal end 12 of the seal 10 can also be employed with either a nipple 6 or conical section 5 designed to prevent deformation in passageway 16, thereby, retaining the sealing characteristics of seal 10.

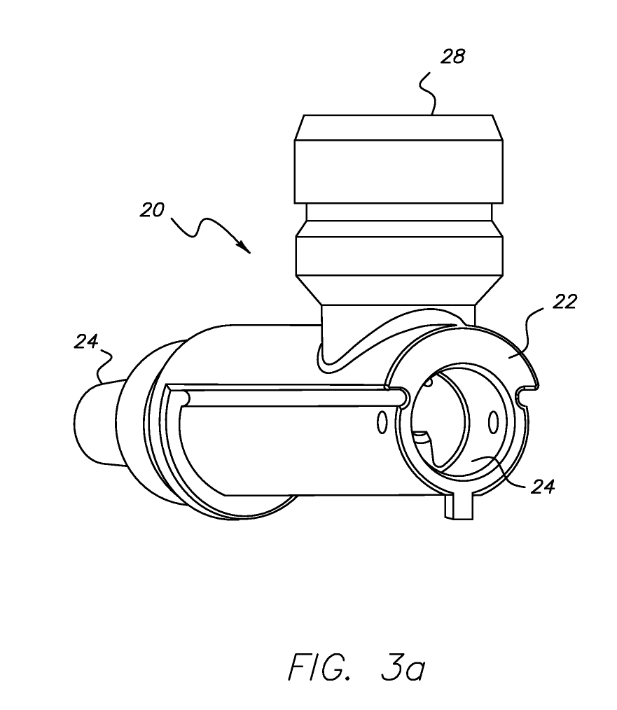

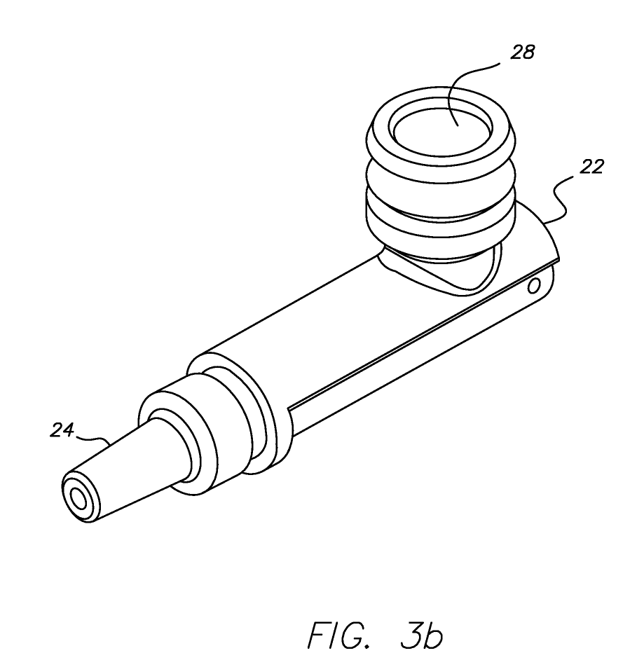

Referring to FIG. 3a, a component in which seal 10 can be used is L-tube 20. The L-tube 20 illustrated has a proximal end 22 and a distal end 24. The proximal end 22 will hold seal 10 within area 24. A needle safety tube (not shown) can be attached to the proximal end 22 of L-tube 20 and seal 10 can maintain a sealing engagement between the L-tube 20 and the needle safety tube. This sealing engagement is maintained while a catheter needle is held within passageway 16 of seal 10 and once the catheter needle is removed from the passageway 16, allowing removal of the catheter needle after canulisation of a patient while preventing fluids from escaping the volume within the L-tube 20 distal from seal 10. The distal end 24 to L-tube 20 is used to hold a catheter assembly to cannulize a patient. Port 28 of L-tube 20 can function as a port to which a luer connection can be attached or a valve. Seal 10 is shaped to allow compress fitting into an area 24 within L-tube 20. The shape of seal 10 assists the closing of passageway 16 after a catheter needle is removed from passageway 16. The seal 10 can have a compliant conical formation that is used to facilitate complete closure. The conical formation permits the use of softer materials while maintaining sealing performance. The uses of softer materials result in less drag allowing the device to function with a lower force spring. Lower force springs are more compact leading to a shorter overall length for the catheter device. The seal is intended to remain in a stationary position once the needle is removed. Using a firmer material for the remainder of seal 10 can hold seal 10 in place within area 24 to facilitate greater stationary positioning. Additionally, a nipple formed on the end of conical section can be made of a softer material or to be moveable and resilient in order to allow the catheter needle to slide while the resiliency of the nipple will close passageway 16 have the catheter needle has been removed.

FIG. 3b gives a perspective illustration of the L-tube 20 from the distal end 24. The distal end of L-tube 20 is closest to the patient and the proximal end 22 is closest to the clinician that is performing the canulation of the patient. The distal end 24 of the L-tube can have a catheter hub attached thereto with a needle used as an introducer into venous tissue of the patient. The proximal end 22 of the L-tube 20 can have a safety tube attached so that the catheter needle can be retracted into the safety tube. The seal 10 can be compress fitted into the proximal end 22 of L-tube 20 with a catheter needle passing through passageway 16. The catheter needle pass through passageway 16 in the center of the seal 10. The seal may be compressed into the proximal end of the L-tube such that a sealing engagement is created between the L-tube and the safety tube. The seal contains a support structure to prevent the passageway within the seal through which the needle traverses from being damaged by the weight of the needle or movement of the needle during retraction. The seal can be designed to fit into the one of the proximal end of the L-tube. The shape and construction of seal 10 allows the passageway 16 through which the needle passes to close after the needle is withdrawn. The seal 10 is shapes to inherently assist the closing the aperture after a needle is removed.

In another embodiment a compliant “nipple” is formed on the conical protrusion 5 10 to facilitate complete closure. The nipple permits the use of softer materials while maintaining sealing performance. The uses of softer materials result in less drag allowing the device to function with a lower force spring. Lower force springs are more compact leading to a shorter overall length for the catheter device. The seal remains stationary within its position in the component as the needle is removed.

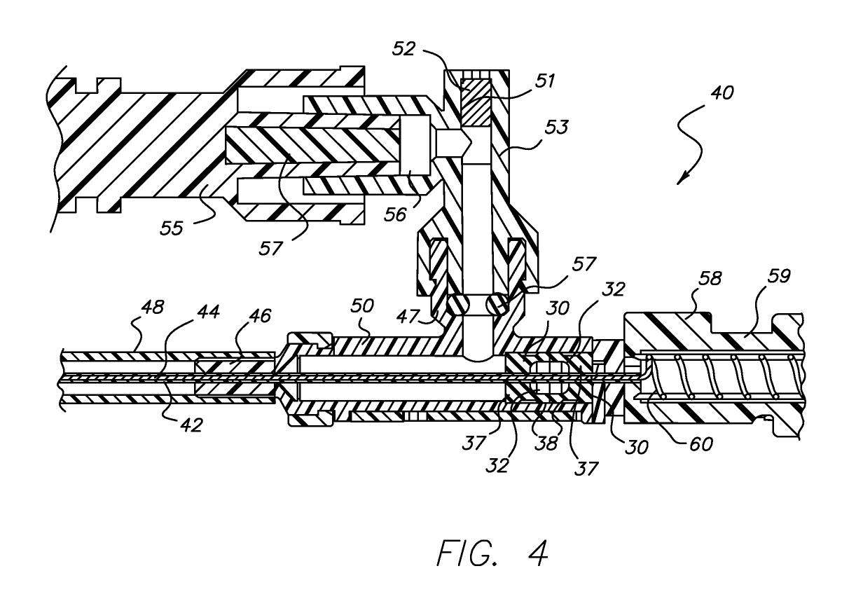

The catheter system 40 in FIG. 4 contains a catheter 42, catheter needle 44 catheter housing 46, safety tube 59 with needle retraction device 60 controlled by actuator 58, L-tube 50, venting mechanism 53 with vent 51 and needle-less valve 55. Seals 50 are contained within L-tube 50 at the proximal end. The needle retraction device 60 is fixedly attached to needle 44 through crimping or other means that allow the needle 44 to be able to slide through seals 30. FIG. 4 illustrates a cross sectional view for an embodiment in which a sealing engagement employs two seals 30 made of an elastomeric material. The elastomeric material can be either silicon, latex or polyisoprene or any combination, thereof, having hardness in a range of 15 to 65 ShoreA. Other embodiments can employ an elastomeric material made from either silicon, latex or polyisoprene or any combination, thereof, with hardness in the range of 35 to 45 ShoreA.

The seals 30 are similar in many ways to seal 10 previously discussed. One basic difference with the seals 30 compared to seal 10 is that seals 30 as shown in FIG. 4 do not contain any conical protrusion 5 that part of seal 10 previously discussed. The absence of conical protrusion 5 is a design choice to reduce drag on needle 44 though the double seals 30 into safety tube 59. Reducing the drag allows a smaller biasing force to be applied by needle retraction device 60 resulting in a smaller overall catheter system 40. Alternative embodiments to that shown in FIG. 4 could have one or both of the seals 30 contain such a conical protrusion 5 and compensate with a larger biasing force in needle retraction device 60. The seal 30 can contain one or more annular ridges 37, 38. Annular ridges 37, 38 apply an axial force due to being pressed fitted inside the proximal end of L-tube 50 allowing seals 30 to create a sealing engagement. The seals 30 each contain a recess 32 that face each other to define an interior volume. Seals 30 remain stationary within their position component as the catheter needle 44 is removed. In FIG. 4, the L-tube 50 has L-tube port 47 positioned in a more distally located position than the single seal 1-embodiment previously discussed wherein port 28 of L-tube 20 was located more proximally. The reason that L-tube port 47 is more distally located than L-tube port 20 is to provide room for two seals 30 instead of a single seal 10. The embodiment in FIG. 4 illustrates two seals 30 that are identical in construction but reversely positioned with recesses 32 facing each other to create an interior volume.

FIG. 4 illustrates a catheter system 40 having catheter 42 and needle 44 formed in a commonly known arrangement as an over the needle catheter system. The catheter 42 is mounted to catheter housing 46 and the catheter needle 44 is in sliding engagement with the catheter housing 46 such that the needle can be retracted into safety tube 59 upon activation of needle retraction device 60 by actuator 58. Seals 30 provide isolation between the internal areas defined by safety tube 59, L-tube 50 and catheter housing 46. Seals 30 are be made from elastomeric materials that allow needle 44 to move through concentric apertures within seals 30 while maintaining sealing engagement with the needle 44.

An embodiment employs L-tube 50 as a catheter component attached to catheter housing 46 at L-tube port 57. The L-tube 50 can be held in a sealing engagement with venting mechanism 53 using o-ring 57 at the junction between L-tube port 47 and venting mechanism 53. The sealing engagement between venting mechanism 53 and L-tube port 47 can be an arrangement so that it is fixedly secured by compatible attachment mechanisms and still remain moveable. For example, an embodiment could have a male attachment mechanism formed at venting mechanism 53 that is compatible with a female attachment mechanisms formed on L-tube port 47. It should be noted that there are many attachment mechanisms that can be used to attach venting mechanism 53 to L-tube port 47 that will be readily apparent to those skilled within the art of catheters.

In an embodiment, venting mechanism 53 is connected to L-tube port 47 using a luer connection. A male luer fitting can be formed on venting mechanism 53 that mates with a female luer connector on the L-tube port 47. Once the luer connection is made, an interior lumen defined by venting mechanism 53 is in fluid communication with the internal volume of L-tube 50. Upon removal of the luer connection, the female luer connector closes, thus sealing the internal volume of the L-tube 50. Venting mechanism 53 has a vent 51 formed therein with a filter 52 placed inside the vent. The function of the vent 51 is to allow air to escape from the internal volume of L-tube 50 and the lumens defined by needle 44 and catheter 42. Design choices for materials used for L-tube 50 can include transparent or translucent materials commonly used with the art of catheters. It is also envisioned that employing a color coded L-tube 50 that identifies that gauge of the luer connection that is to be used with L-tube 50.

Flashback can be controlled by placement of a venting mechanism within a component that is to be used in conjunction with the catheter 42. The placement of vent 51 in an area separate from the catheter housing 46 and L-tube 50 provides an area that can be arranged independently from the positioning of catheter housing 46 and therefore, specifically arranged for observing and controlling Flashback. The venting is controlled by having a vent 51 allow air with the lumens defined by needle 44 and those lumens within fluidly connected components to vent air from the catheter assembly 40. Once Flashback occurs, the blood pressure from the vena-puncture will force air through the vent 51 until blood reaches the filter plug 52. The filter plug 52 can be formed from a hydrophobic or other material that will allow air to pass but not fluid. The Flashback is controlled using the filter 52 within vent 51 to allow air contained within the various fluidly connected lumens to escape until blood reaches filter 52 resulting in the stoppage of flow through vent 51. Thus, air originally contained within the lumens defined by needle 44, catheter housing 42 and the internal volume of L-Tube 50 passes through vent 51. Proper positioning of vent 51 during canulation will allow the all the air within the lumens defined by needle 44, catheter housing 52 and the internal volume of L-Tube 50 to escape. Once the air has vent out, the blood from the Flashback will be forced upon the interior region of the filter 52 and stop venting through vent 51.

FIG. 4 further illustrates a needle-less valve 55 connected to second port 56 on venting mechanism 53. As shown, second port 56 is constructed as a female luer connector; however, many alternative arrangements will be readily apparent to those skilled in the art. The needle-less value 55 has a compatible male luer 57 connector to connect to the female luer connector configuration of second port 26. Placement of the vent 51 near or adjacent the second port 56 allows virtually all the air contained in the interior spaces defined by the lumens defined by needle, 44, catheter 42, catheter housing 46 and internal volume 45 (which includes the volume near the second port 56) to be evacuated upon Flashback. Examples of sources for needle-less valves are Alaris®; Cardinal Health®; Rymed Technologies, Inc.; Creative Plastic Technology, LLC; ICU Medical, Inc.; B. Braun Melsungen AG; and various others that will be readily apparent to those skilled in the art.

FIG. 4 illustrates catheter system 40 having a catheter 42 and needle 44 attached to a catheter housing 46. Safety cap 48 can be removed exposing needle 44 allowing vena penetration of a patient. Once canulation is completed, actuating device 58 retracts needle 44 into safety tube 59. Needle retraction device 60 can be a biasing mechanism such as a spring or other biasing device that is securely held in place inside safety tube 59. Actuator 58 enables the biasing force of needle retraction device 60 to pull needle 44 into safety tube 59.

1. A sealing engagement within a catheter system comprising:
an elastomeric material with a hardness in a range of 15 to 65 ShoreA used to form said sealing engagement, said elastomeric material having a proximal end and a distal end;
a firm cylindrical object having a predetermined cross-section slideably mounted within a passageway in said elastomeric material, said passageway within said elastomeric material traversing from said distal end to said proximal end of said elastomeric material;
an annular area formed near at least one of said proximal end and said distal end of said elastomeric material, said annular area having an outside diameter defining a ridge; and
a recess formed on at least one of said proximal end and said distal end, said recess defining a cavity.

2. The sealing engagement of claim 1 further comprising a compliant protrusion formed within said recess.

3. The sealing engagement of claim 2 wherein said elastomeric material further comprises said compliant protrusion and said ridge on said annular area being formed on the same end, and wherein said elastomeric material is placed within a catheter component of a predetermined sized and shape such that said ridge on said annular area acts to compress said compliant protrusion closing compliant protrusion around said interior passageway once said firm cylindrical object is removed from said passageway.

4. The sealing engagement of claim 3 wherein said firm cylindrical object is a catheter needle and said elastomeric material remains stationary within its position in said component as the catheter needle is slide within said passageway and removed.

5. The sealing engagement of claim 4 wherein said elastomeric material forms said sealing arrangement between said component and a safety tube, wherein said safety tube has a retraction device to bias said catheter needle and retract said catheter needle into said safety tube upon actuation.

6. The sealing engagement of claim 5 wherein said compliant protrusion is made from a softer material than said elastomeric material, said softer material imparting less drag on said catheter needle when said catheter needle is retracted into said safety tube.

7. The sealing engagement of claim 1 wherein said elastomeric material further comprises a first seal and a second seal with each said first seal and said second seal containing said ridge on said annular area at the same said end as said recess, and wherein said first seal and said second seal are arranged within said catheter component such that said recesses face each other to define a volume between said first seal and said second seal.

8. The sealing engagement of claim 1 further comprising at least one support structure formed at the proximal or distal end of said elastomeric material to prevent deformation of said interior passageway.

9. The sealing engagement of claim 1 wherein said elastomeric material comprises

10. The sealing engagement of claim 1 wherein said elastomeric material has a hardness in a range of 35 to 45 ShoreA.

11. A sealing engagement within a catheter system comprising:
an elastomeric material having a cylindrical shape and a hardness in the range of 15 to 65 ShoreA, said elastomeric material being fitted into a catheter component to form said sealing engagement, said elastomeric material having a proximal end and a distal end;
a firm cylindrical object having a predetermined cross-section slideably mounted within a passageway in said elastomeric material, said interior passageway traversing the longitudinal axis of said cylindrically shaped elastomeric material from a distal end of said elastomeric material to a proximal end of said elastomeric material;
at least one annular area formed near one of said ends, said annular area having an outside diameter defining a ridge such that said elastomeric material exerts a compression force onto said passageway inside said catheter component; and
a recess formed within said elastomeric material at any of said ends defining of said ridge.

12. The sealing engagement of claim 11 further comprising a compliant protrusion formed within said recess such that said protrusion has a center that is concentric to the longitudinal axis of said cylindrically shaped elastomeric material and said passageway passes through said center of said compliant protrusion, said compliant protrusion being formed at any of said ends where said ridge on said annular area is formed such that said ridge on said annular area acts to compress and close said compliant protrusion around said interior passageway once said firm cylindrical object is removed from said passageway.

13. The sealing engagement of claim 12 wherein said elastomeric material comprises silicon polyisoprene.

14. The sealing engagement of claim 11 wherein said firm cylindrical object is a catheter needle and said seal remains stationary within its position in said component as the catheter needle is removed.

15. The sealing engagement of claim 14 wherein said elastomeric material forms said sealing arrangement between said component and a safety tube, wherein said safety tube has a retraction device to bias said catheter needle and retract said catheter needle into said safety tube upon actuation of said retraction device.

16. The sealing engagement of claim 14 wherein said compliant protrusion is made from a softer material than said elastomeric material, said softer material imparting less drag on said catheter needle when said catheter needle is retracted into said safety tube.

17. The sealing engagement of claim 11 wherein said elastomeric material further comprises a first seal and a second seal with each said first seal and said second seal containing said ridge on said annular area and a single said recess in said elastomeric material at one of said ends that defines said ridge, wherein said first seal and said second seal are arranged within said catheter component such that each respective said recess face each other defining a volume between the first and second seal.

18. The sealing engagement of claim 11 wherein said elastomeric material has a hardness in a range of 35 to 45 ShoreA.

19. A method of forming a sealing engagement within a catheter system comprising the steps of:
forming an elastomeric material of either silicon and/or polyisoprene into a cylindrical shape such that there is at least one annular area formed near an end of said elastomeric material, said annular area having an outside diameter defining a ridge, and forming a recess within said elastomeric material at said end defining said ridge, said elastomeric material having a hardness in the range of 15 to 65 ShoreA;
placing said elastomeric material into a catheter component such that said ridge exerts a compression force onto said catheter component;
inserting a catheter needle through a passageway along the longitudinal axis of said cylindrically shaped elastomeric material; and
retracting said catheter needle through said passageway, wherein said compression force forces said passageway to close creating said sealing engagement.

20. The method of claim 19 further comprising the step of: forming said passageway prior the step of be either: pushing said catheter needle through said elastomeric material; preforming said passageway by cutting using a single edge pin like cutting instrument; or preforming said passageway y cutting using a trocar tipped edge.
